(19) United States
(12) Patent Application Publication
(10) Pub. No.: US 2005/0260281 A1
(43) Pub. Date: Nov. 24, 2005

(54) Upper respiratory tract virus infection preventive therapy

(76) Inventor: Thyparambil Chacko Mathew, Louisville, KY (US)

Correspondence Address: THYPARAMBIL CHACKO MATHEW, 4521 WOLFCREEK PARKWAY, LOUISVILLE, KY, 40241, US

(21) Appl. No.: 10/847,961

(22) Filed: May 19, 2004

(51) Int. Cl. A61K 33/36

(52) U.S. Cl. 424/671; 424/682

(57) ABSTRACT

This upper respiratory tract virus preventive therapy, also called MGM therapy, involves gargling with 140-145 F hot, 5% iodized common salt solution, followed by gargling with approximately the same temperature hot water (without salt in it). The therapy calls for repeating the procedure at every 4-5 hours, until the symptoms cease to manifest. The therapy is intended for preventing viral infection such as ‘Common cold’ through URT, as well as preventing lung infection that usually follows a viral infection.

### BACKGROUND OF THE INVENTION

This invention is about a powerful therapy which is affordable, as well as simple in application to prevent or minimize the attack of numerous Common Cold and other Viruses that try to get into our system through upper respiratory tract (URT) The method also applies to minimizing the effect of Tonsillitis and Sinusitis.

This invention is an optimized version of the centuries old “Grandma Method” (GM). Therefore, the Inventor prefers to call the therapy, MGM (Modified Grandma Method). In real life practice of MGM therapy by the inventor for over 20 years, along with willing family members, numerous friends, relatives and others in the United States and in India, inventor has concluded that MGM offers an extremely effective tool to prevent LUNG INFECTION from any viruses possibly including the current world wide threat of SARS as long as the viruses enter into the body through the membranes of the Upper Respiratory Tract (the nose, nasal passages and the throat).

What was wrong with the “grandma recipe” of common cold prevention and treatment? Gargling with luke-warm water salt solution was the recipe of Grandma's around the world. It definitely helped to some extent, in cases where the virus involved was not very powerful. However, sore throats, runny noses, lose of voice function, lung infections, along with coughing appeared in practically everyone affected with the variety of common cold viruses all over the world. GM failed quite often especially when the strain involved was strong. Therefore, many who practiced GM lost faith in it and naturally discontinued doing it or did not bother to do it.

Another important reason for the failure of GM was the notion that you would only do GM if you experience pain in the throat region. The de-sensitization of the throat area due to severe viral infection was not recognized in the GM therapy.

### MGM and the Results

MGM therapy has been found to be effective in actually destroying the viruses or preventing the spread of infection to the lungs. The therapy also sustains relief of the symptoms associated with the URT viral infection, allowing only a minimum disruption of daily activities.

### DETAILED DESCRIPTION OF MGM RESULTS

The therapy prevents sore throat (tracheitis) and loss of voice (laryngitis). The therapy eliminates or minimizes the need for coughing, running nose, stuffy nose and lung infection; thereby preventing complicated pneumonia. The greatest benefit of the method is preventing others from contracting the disease especially family members. However, it requires prompt action especially because the symptoms of the disease are normally revealed after a day or two of viral migration into the body, usually through upper respiratory tract (URT).

MGM therapy works well if one can recognize the initial symptoms early; such as a slight irritation in the throat area, slight changes in your nasal area and is willing to carry out MGM therapy promptly. MGM therapy takes only five minutes to do and the reward is: prevention of viral infection.

### Illustration with a Case History

The patient, an M.D., although well aware of the benefits of MGM, never cared to do the therapy promptly or correctly. Five years ago she ran into big trouble. Viral infection started as a “common cold”, soon elevated into pneumonia within a few days. Her condition deteriorated rapidly and she was admitted into the hospital for ten days or so. Every known test was preformed. When she was released from the hospital, she was extremely weak, partly due to the medications she had to take and partly due to the severity of the lung infection. It took more than three months before she could return to work. The final conclusive report of her physician was that, the patient did allow a common cold virus go unchecked. Ever since the episode, she has became an ardent believer in MGM therapy and practices MGM routinely and never had the same out of control viral infections again so far.

In the meantime, the virus failed to affect the husband of the patient even though he slept in the same bed during her illness and attended to her throughout the whole ordeal. Although he husband had light symptoms of a common cold during that period, the virus never got an opportunity to affect his lungs nor irritate his nasal and throat areas. He did MGM therapy three to four times a day when his wife was at home, and once a day when she was admitted into the hospital. This incident is verifiable.

### MGM Therapy Procedure

1. Take approximately half a cup of hot water (200 ml) approximately 140 to 145 degrees F. (60-66 C). This temperature is a slightly higher temperature than that of freshly poured coffee. (Use a kitchen thermometer to check the temperature)
2. Add a teaspoon full of common salt (8-10 gms) and stir well with the spoon to dissolve. Have another cup of “coffee hot” water (without salt) ready.
3. Gargle a minimum of three times with the hot salt water.
   Cautions:
A. For each gargling, take enough hot salt water into the mouth to cover the throat area (approximately 40 ml.)
B. During gargling the neck needs to be tilted back as far as possible for effective coverage of the solution.
C. Each gargling attempt should last 5 to 7 seconds, and then, promptly spit the mouth contents into the sink (preferably with water running).
4. After gargling 3 times with hot salt solution, gargle with hot water (without salt) preferably the same temperature (140 to 145 F) at least three times in a similar way as described above.
5. This is followed by cleaning up all the salt residues from the mouth areas by swishing around with warm water (<130° F.) several times.

If MGM therapy is done correctly, all the stuffy gunk in the nasal area will automatically emerge out (as if, the person went through a “steam inhaling technique”) and it is important to clean the nasal areas, face etc. thoroughly with warm water right away.

Additional Cautions and Information:
A. Care must be exercised not to burn up the throat area by using very hot (over 145° F.). If you dip your finger in the water and if you pull out the finger instantly, the water is too hot. If the finger can remain in the water for more than one second, it is likely to be below 140° F.
B. Iodized salt is preferred because of the expectorant use of potassium iodide.

### GM Vs. MGM

Both procedures recognize the powerful destructive effect of the chemical Sodium Chloride (common salt) on microbes, viruses and bacteria. GM does not recognize the effect of temperature. MGM strongly recommends specific optimum temperature 1400 to 145° F. (40° to 140° F. was long recognized as the ideal condition for the growth of microbes and bacteria.)

GM did not specify the final concentration of common salt in the gargling solution where as MGM recommends specific concentration to optimize the result.

GM does not insist on a time when to start the procedure whereas the “time to start” the procedure is the centerpiece of MGM therapy which is “earlier the better”.

### Commencement and Intended Aims of MGM Therapy

The whole idea of MGM therapy is to prevent lung infection that usually follows a mishandled viral infection through URT. MGM reduces the chance for lung infection, pneumonia, high fever etc. If waited too long after the symptoms of throat pain appeared, the lungs would be infected. And obviously, one has to resort to medical treatment under the direction of a doctor.

Daily practice of MGM therapy is required if viral infections are prevalent in the area. If MGM therapy is commenced at the very onset of symptoms such as very slight irritation in the nasal and throat area and if the therapy is continued every three hours or, twice a day, the lung infection is not likely to happen. If the strain is very powerful, the symptoms could reappear within two to three hours after doing MGM therapy; but at much reduced strength each time, while continuing to MGM therapy for two to three days.

### Prevention Versus Treatment

Everyone prefers prevention of disease. Prevention requires some important discipline by the individual, the society as a whole and by government agency directives.

Individual Discipline: One cannot be a procrastinator to take prompt actions as required. Without this discipline, preventive therapies are likely to fail. For example: MGM therapy will work as intended only if done promptly at the slight appearance of symptoms.

All established individual hygiene procedures must be practiced at home. CDC guidelines will help. Some of them are: Clean up, hands, face etc. often with detergent soap and apply moisturizers; drink small quantities of clean water more often than usual; Always cover face when sneezing, coughing etc. preferably with a clean handkerchief, towel or disposable paper napkin etc.; Equally important is to dispose of soiled napkins etc. into designated bin; use cloth napkins in a common sense way, by using a different fold and by removing soiled ones promptly for laundering; Use of a humidifier (of any kind), placed near the affected person could help for it has been well established that viruses enter into the cells when the nasal area is too dry.

### Suggested Government Action

Encourage the practice of MGM for all citizens as a public policy facilitating practice of MGM therapy in public places such as in restrooms of airports, bus stations, schools, convention halls and other places.

### Mechanism of MGM Therapy

The effectiveness of the therapy is indeed due to the optimization of the concentration of salt solution (5%) as well as the temperature of the solution (140-145 F). These optimum conditions facilitated the penetration of the salt deep into the cell wall. (In GM therapy, since the concentration and the temperature of the salt solution used generally have been much lower which resulted in less penetration of the salt to the cell wall of the throat areas and consequently GM therapy failed to ward off viral attack through URT in most cases). The above mechanism of MGM therapy is as follows: After gargling 3 times with salt solution according to the procedure recommended, followed by gargling with almost same temperature water (140-145 F), the patient would not experience salt ‘after taste’, where as, if the plain water gargling was done 3 times with just warm water, the patient will experience salt after taste for a while (>5 minutes). Obviously, the warm water used, failed to extract all the salt that penetrated into the cell wall which would explain the after taste of salt several minutes after the procedure.

1) Approximately 5% iodized common salt concentration (4 to 6%) water solution at 140 degree F. (138 to 145 F) results in an optimum condition for effective annihilation of the viruses entering through URT.

2) If MGM is practiced a minimum of two times a day at the very onset of slight symptoms, the symptoms totally disappear in less than three days.
2A) The patient would not lose voice at in most cases
2B) The patient would stop sneezing or would experience much reduced sneezing.
2C) In most cases, the patient would not have “runny nose” symptom as well as congestion of the nose.

3) If MGM therapy is commenced (every 2 to 3 hours) after fully blown symptoms (except lung infection) are displayed by the patient, lung infection may not occur or the lung infection if occurs, would be less severe.
3A) The patient's lost voice would be promptly restored and maintained.
3B) De-sensitized throat area will be sensitized.
3C) Nose congestion and “runny nose” would ease considerably.
3D) No groggy feeling would be experienced at anytime.

4) If MGM therapy is practiced every 2 to 3 hours, recovery time would be reduced.
4A) Nasal congestion will ease considerably and “runny nose” would disappear at this stage of infection.

5) The viral infection would stop transmitting to others even without the use of a facemask, if MGM is practiced at the very onset of symptoms and continued until symptoms have totally disappeared. (This occurs due to the reduction or strength of the viruses due to the absence or reduction of sneezing, runny nose etc.)

6) Travelers would not feel giddy or groggy. If MGM therapy is practiced promptly and repeated quite often as recommended, infected individuals would not serve as transmitters of the disease for the same reasons mentioned in claim number five.
